(19) United States
(12) Patent Application Publication
(10) Pub. No.: US 2025/0318770 A1
(43) Pub. Date: Oct. 16, 2025

(54) METHODS AND SYSTEMS FOR ASSESSING NEUROLOGICAL CONDITIONS

(71) Applicant: Axon Interfaces Corp., Palo Alto, CA (US)

(72) Inventors: Soren Madsen, San Francisco, CA (US); Julian Marc, Palo Alto, CA (US); Malcolm McSwain, San Francisco, CA (US)

(21) Appl. No.: 19/170,334

(22) Filed: Apr. 4, 2025

Related U.S. Application Data
(60) Provisional application No. 63/632,341, filed on Apr. 10, 2024.

(51) Int. Cl. A61B 5/372; A61B 5/00; G16H 50/20

(52) U.S. Cl. CPC A61B 5/372; A61B 5/7264; G16H 50/20

(57) ABSTRACT

Methods and systems for assessing neurological conditions. Raw EEG resting state data is obtained from a patient and filtered. The filtered EEG data includes signals in a plurality of channels. Bad channel data and large artifacts are removed and biomarkers including spectral features, statistical features, time series features, and graph features are extracted from the filtered EEG data. The extracted biomarkers are provided as inputs to a machine learning engine that has been trained to distinguish disease states from non-disease states. The machine learning engine outputs a result as a probability from 0 to 1 of the presence of a disease state as indicated by the extracted biomarkers.

### RELATED APPLICATIONS

This is a non-provisional and claims the priority benefit of U.S. Provisional Application No. 63/632,341, filed Apr. 10, 2024, which is incorporated herein by reference.

### FIELD OF THE INVENTION

The present invention relates to methods and systems for assessing neurological conditions.

### BACKGROUND

EEG tests are commonly used to determine overall electrical activity of the brain (for example, to evaluate trauma, drug intoxication, brain damage, etc.) and/or to evaluate brain disorders. For example, seizure activity due to epilepsy typically presents as rapid spiking waves on an EEG trace. Patients with brain lesions may produce atypically slow EEG waves. Other brain disorders may also be associated with characteristic EEG patterns.

An EEG test is typically administered by placing electrodes consisting of small metal discs with thin wires onto a patient's scalp. The electrodes detect electrical signals that result from brain cell activities, and these signals are subsequently amplified before being displayed on a computer display screen and/or printed on a chart. It then typically falls to a specialist to interpret the results by inspecting the displayed or printed signals.

### SUMMARY OF THE INVENTION

Methods and systems for assessing neurological conditions. In one embodiment, raw electroencephalogram (EEG) resting state data is obtained from a patient, e.g., using one or more sensors affixed to the patient or a device worn by the patient. The raw EEG data is band pass filtered to obtain filtered EEG data made up of signals in a plurality of channels. Bad channel data and large artifacts are removed from the filtered EEG data so as to eliminate or reduce signals in channels that exhibit poor signal-to-noise ratios (e.g., those signals which in the time domain have channel amplitudes that deviate by more than a specified amount and/or those signals which in a frequency domain exhibit unwanted deviations in channel spectral data) and signals from electrical sources other than the patient's brain activity. Biomarkers including spectral features, statistical features, time series features, and graph features are extracted from the filtered EEG data, and the extracted biomarkers are provided as inputs to a machine learning engine that has been trained to distinguish disease states from non-disease states. The machine learning engine outputs a result as a probability from 0 to 1 of the presence of a disease state as indicated by the extracted biomarkers.

In various embodiments, the bad channel removal may. be performed using one of: a k-nearest neighbor approach, anomaly detection, or a Local Outlier Probability (LoOP) approach. The large artifact removal may be performed by eliminating channel data that exceeds a predefined amplitude threshold, or by one or more of: regression methods, wavelet transformation methods, Blind Source Separation (BSS)-based methods, or Empirical-mode Decomposition (EMD) methods. The feature extraction may be performed using one or more of: Fast Fourier Transforms (FFTs), discrete wavelet transform (DWT), time frequency distributions, eigenvector methods, or autoregressive methods.

In various embodiments, the spectral features may include alpha peak frequency, band power, and delta/theta power ratio. The statistical features may include Hjorth activity, Hjorth mobility, and Hjorth complexity. The time series features may include normalized Lemple-Ziv complexity and fractal dimension. And the graph features may include mean jump length across a quantile graph (Delta score).

The machine learning engine may be instantiated as computer system-based instance of a software-as-a-service platform or as a component of a wearable device. For example, such a wearable device or computer system may include a processor and a memory coupled to the processor, the memory storing instructions which, when executed by the processor, cause the processor to execute the steps for processing raw EEG resting state data as recited above and further described herein.

### BRIEF DESCRIPTION OF THE DRAWINGS

The present invention is illustrated by way of example, and not limitation, in the figures of the accompanying drawings, in which:

FIG. 1 is a flow diagram illustrating a process for pre-processing of raw EEG resting state data to enhance the analysis of biomarkers related to one or more neurodegenerative diseases in accordance with one embodiment of the present invention.

FIGS. 2A and 2B are examples of raw EEG data; in FIG. 2A the amplitudes of several channels of data are plotted as a function of time; in FIG. 2B similar data is plotted in the frequency domain.

FIG. 3 shows an example of a sensor arrangement for collection of EEG data in a 19-channel configuration.

FIGS. 4A and 4B illustrate examples of filtered EEG data; in FIG. 4A the amplitudes of the channels of filtered EEG data are plotted as a function of time; in FIG. 4B similar filtered EEG data is plotted in the frequency domain.

FIG. 5 shows a table of extracted biomarkers from channels of EEG data in accordance with one embodiment of the present invention.

FIG. 6 shows an example of a wearable device configured to capture EEG data and perform pre-processing in accordance with one embodiment of the present invention.

FIG. 7 shows an example of a wearable device in the form of headband with sensors tethered by wires in accordance with one embodiment of the present invention.

FIG. 8 illustrates an example of a system for providing software-as-a-service access for assessing neurological conditions from EEG data in accordance with one embodiment of the present invention.

FIG. 9 is a flow diagram illustrating one example of a user session for the system shown in FIG. 8.

FIG. 10 shows an example of a computing system suitable for use in connection with embodiments of the present invention.

### DETAILED DESCRIPTION

In the following detailed description of presently preferred embodiments, reference is made to the accompanying drawings that form a part hereof, and in which are shown by way of illustration specific embodiments in which the invention may be practiced. It should be understood that other embodiments may be utilized and structural changes may be made without departing from the scope of the present invention. Descriptions associated with any one of the figures may be applied to different figures containing like or similar components/steps.

In accordance with one embodiment of the invention, raw electroencephalogram (EEG) resting state data is pre-processed to enhance the analysis of biomarkers related to one or more neurodegenerative diseases, for example, Alzheimer's disease. Pre-processing of the raw EEG data provides engineered EEG data that is different from the raw data in that it presents the desired biomarkers in a manner that they are more-readily assessed than in the raw data. The pre-processed data is then analyzed to determine whether a disease state is present or not. Additional embodiments of the invention provide an EEG data-analysis-on-demand platform that is accessible over a computer network and which provides analysis of EEG data, either live or pre-recorded, in accordance with the present methods. And, still further embodiments of the invention provide a data collection platform in the form of an instrumented, head-worn data collection device that may be configured for on-device data analysis and/or data download in accordance with the present methods.

As noted above, EEG tests are commonly used to determine overall electrical activity of the brain. However, conventional EEG tests typically produce results that are displayed on a computer display screen and/or printed on a chart, and which require interpretation by a specialist. This process is time consuming and often presents inconveniences for a patient.

One aspect of the present invention is that the present methods and system allow healthcare providers rapid (near immediate) access to analysis of EEG tests. Rather than having to refer patients to a specialist, a provider is able to access a web-based platform designed to enhance EEG devices with the capability to assess neurological conditions from resting-state data. By analyzing a patient's brain's electrical activity, this platform extracts biomarkers that enable the analysis of neurodegeneration. This approach affords the provider a direct, user-friendly means to assess brain health and function across a myriad of conditions. In some cases, for example where web access is unavailable or cost-prohibitive, the platform may be instantiated, in-full or in-part, in a head worn device. Or, a sensor and data collection/pre-processing component may be instantiated in the head-worn device, with final analysis of the pre-processed data being performed by a computer-based component after the pre-processed data is downloaded from the head-worn device. Other embodiments of the invention will also be apparent from the description provided below.

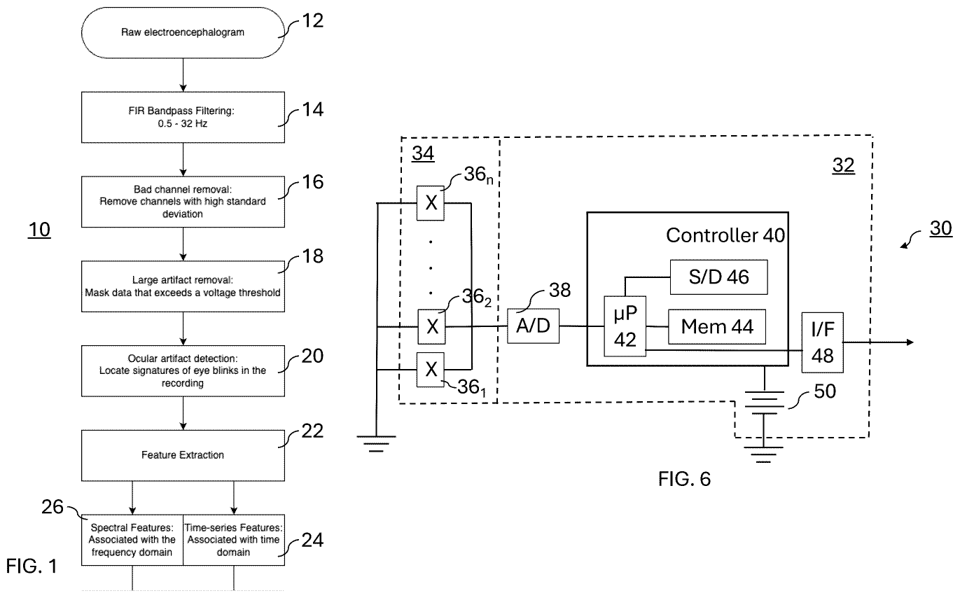

Referring now to FIG. 1, one embodiment of a process 10 for pre-processing of raw EEG resting state data to enhance the analysis of biomarkers related to one or more neurodegenerative diseases is illustrated in the form of a flow diagram. At 12, the raw EEG data is obtained. The data may be obtained live from one or more EEG sensors affixed to a patient, or it may be obtained from a stored replica of such data, e.g., a recording of live data that was preserved for later pre-processing and analysis. In practice, the EEG data may be obtained from the patient by affixing one or more EEG sensors in the form of metal discs with thin wires running therefrom onto a patient's scalp. Or, in some instances, the EEG data may be obtained from a head-worn device that includes sensors of this kind. Discussed further described below is an example of a head-worn device that allows on-board pre-processing of the EEG data in accordance with process 10 and, optionally, analysis of the pre-processed EEG data.

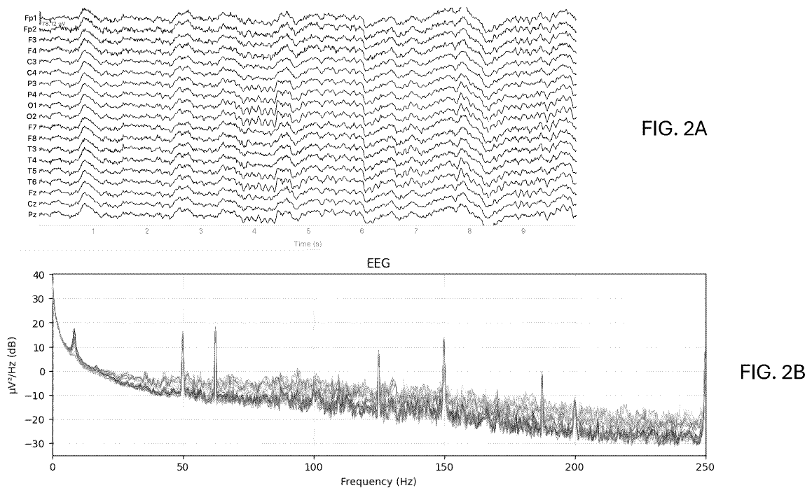

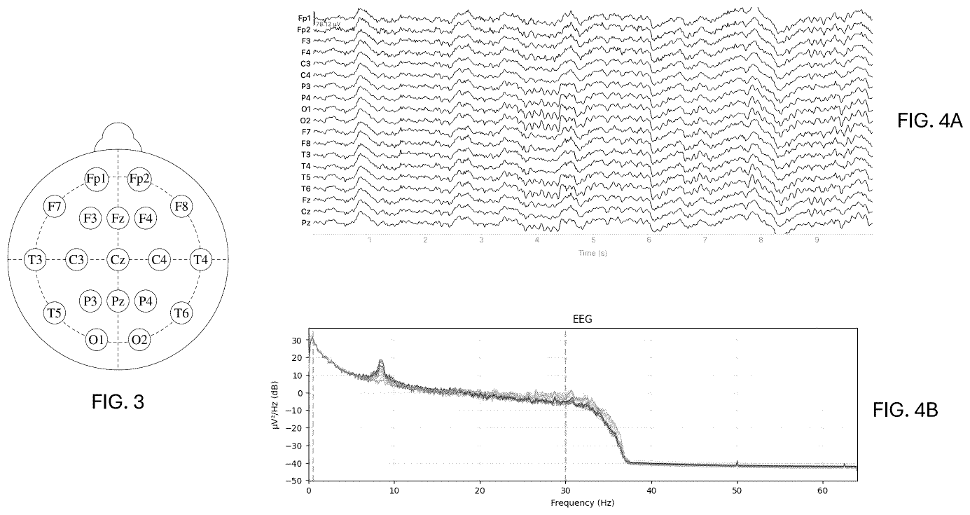

The raw EEG data may resemble that illustrated in FIGS. 2A and 2B. In FIG. 2A, the amplitudes of several channels of data are plotted as a function of time. FIG. 2B shows similar data plotted in the frequency domain. Although 19 channels worth of data are illustrated, in other embodiments 32 channels, 64 channels, or other numbers of channels of data may be captured by the EEG sensors. In the 19-channel configuration, sensors are placed over the frontal, central, parietal, and occipital regions of the scalp in a conventional arrangement, as illustrated in FIG. 3. The 19 channels of data are: Fp1, Fp2, F3, F4, C3, C4, P3, P4, O1, O2, F7, F8, T3, T4, T5, T6, Fz, Cz, and Pz. In a 64-channel EEG setup, additional sensors are distributed across the scalp to cover regions and capture signals that may otherwise be missed in a 19-channel configuration.

Returning to FIG. 1, the raw EEG data is band passed filtered at step 14. The raw EEG data typically consists of signals in the range 0.01 Hz to approximately 100 Hz. In one embodiment, a finite impulse response (FIR) bandpass filter may be used, with a passband of approximately 0.5-32 Hz, so that the filtered EEG data is restricted to signals in the pass band range. In other embodiments, narrower or wider bandpass filters may be used to eliminate undesired frequency components. The filtered EEG data may resemble that illustrated in FIGS. 4A and 4B. In FIG. 4A, the amplitudes of the channels of filtered EEG data are plotted as a function of time. FIG. 4B shows similar filtered EEG data plotted in the frequency domain.

Following the bandpass filtering, the now-filtered EEG data is subjected to bad channel removal 16. At this stage of the process channels with poor signal-to-noise ratios are removed. Bad channels may be due to a variety of factors, such as poor electrical connections to the sensors, signals from electrical sources other than brain activity, electromagnetic interference from non-patient sources, etc. So-called bad channels typically exhibit higher noise content than other channels and so including the data from bad channels may improperly influence decisions based on the data. By removing the bad channels, more accurate decision making is possible.

Bad channel data can be identified in either, or both, the time domain and/or the frequency domain. In either instance, channel data that deviates by a specified amount (e.g., exhibits a standard deviation exceeding a threshold value) may be used to identify bad channels. For example, in the time domain, channel amplitudes that deviate by more than approximately 100 microvolts may be used to identify bad channels. In the frequency domain, deviations identified in channel spectral data may be used to identify bad channels. Among the techniques that may be employed for bad channel detection and removal are a k-nearest neighbor approach as proposed by Ramaswarmy et al., “Efficient Algorithms for Mining Outliers from Large Data Sets,” SIGMOD Rec. 2000, 29, 427-438, anomaly detection according to a Local Outlier Factor, as proposed by Breunig et al., “LOF: Identifying Density-Based Local Outliers,” SIGMOD Rec. 2000, 29, 93-104, a Local Outlier Probability (LoOP) approach as proposed by Kriegel, et al., “LOOP: Local Outlier Probabilities,” Proc. 18th ACM Conf. on Information and Knowledge Management (CIKM '09), Hong Kong, China, 2-6 Nov. 2009; ACM (2009), pp. 1649-1652, or another approach.

Following bad channel removal, additional large artifact removal 18 may be performed. As mentioned, artifacts due to signals from electrical sources other than brain activity, e.g., blinking, cardiac activity, etc., may be present in the channel data and should be removed as part of the pre-processing activity. This may be done, for example, by eliminating channel data that exceeds a predefined amplitude threshold. Other methods of artifact removal may also be performed (or performed in lieu of thresholding), for example, regression methods, wavelet transformation methods, Blind Source Separation (BSS)-based methods, Empirical-mode Decomposition (EMD) methods, or others. In some cases, ocular artifact detection and removal may be performed as a separate step 20, using any of the aforementioned techniques.

Following the artifact removal, feature extraction 22 may be performed. This may be done using approaches such as Fast Fourier Transforms (FFTs), discrete wavelet transform (DWT), time frequency distributions, eigenvector methods, autoregressive methods, or others. The features of interest may be associated with the time domain 24 and/or the frequency domain 256. Feature extraction provides dimensionality reduction and data compaction, allowing for efficient use of machine learning approaches for data classification; that is, determination of whether a disease state is present or not.

In one embodiment of the present invention, the following features are computed for each channel of EEG data. We refer to these features as biomarkers.
a. Spectral Features:
   Alpha peak frequency
   Band power
   Delta/Theta power ratio
b. Statistical Features:
   Hjorth activity
   Hjorth mobility
   Hjorth complexity
c. Time-Series Features
   Normalized Lemple-Ziv complexity
   Fractal dimension
d. Graph Features
   Mean jump length across a quantile graph (Delta score)

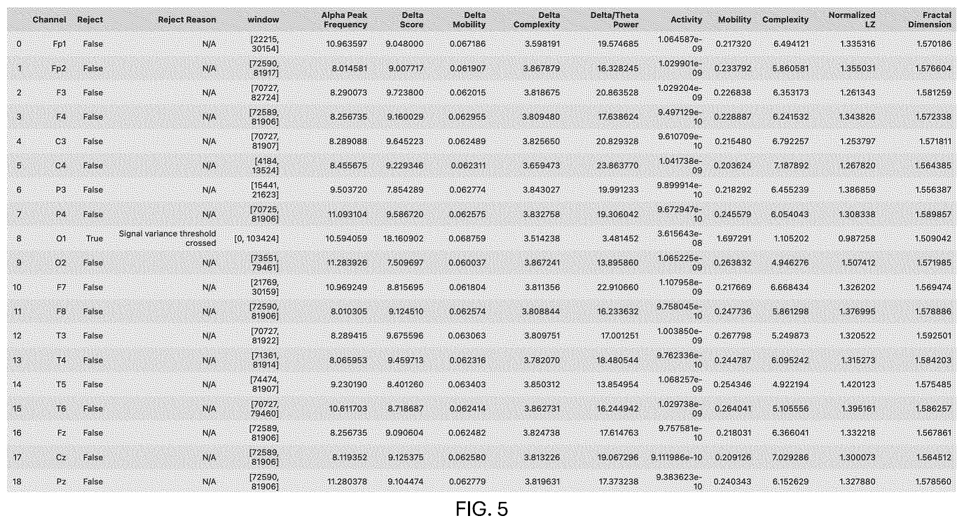

FIG. 5 shows a table of extracted biomarkers from channels of EEG data in accordance with the present method. The extracted biomarkers may be used as inputs to a machine learning engine that has been trained to distinguish disease states from non-disease states according to the extracted biomarkers. The machine learning engine may be instantiated as a server instance of a software-as-a-service platform, as described further below. In one embodiment, the machine learning engine is trained to determine the presence of Alzheimer's disease from a disease-free condition based on the biomarkers. The biomarkers extracted from the EEG data are applied as inputs to the trained machine learning engine and a probability from 0 to 1 of the presence of a disease state is provided as an output. The machine learning engine will preferably have been trained using labeled data so that the accuracy of a disease state prediction is enhanced. Alternatively, unsupervised learning (that is training without labeled data) may be employed, however, the efficacy of a machine learning engine trained in such an approach may not be suitable for clinical purposes.

Referring now to FIG. 6, an example of wearable device 30 configured to capture EEG data and perform the pre-processing described above is illustrated. Wearable device 30 includes an electronic controller section 32 and a head-worn sensor section 34. The head-worn sensor section 34 is adapted to fit over the head of a patient and is fitted with a plurality of sensors 36₁, 36₂, . . . 36ₙ, that are responsive to electrical activity of the brain to produce signals that are passed along thin wires to an analog to digital converter 38 of the electronic controller section 32. Although not shown, before being digitized, the signals from the sensors 36₁, 36₂, . . . 36ₙ may be amplified and/or recorded in analog form. Electronic controller section 32 includes a programmable unit (e.g., a processor, controller, or similar device) 40 that is communicably coupled to A/D converter 38 to receive the digitized signals produced by the one or more sensors 36₁, 36₂, . . . 36ₙ, and perform the pre-processing described above. In some instances, the programmable unit 40 may further be configured to determine whether or not those signals correspond to a disease state or not. The programmable unit 40 includes a microprocessor or similar unit 42 and a memory 44 coupled thereto, which may be used to store copies of the sampled EEG signals as well as intermediate program results. And, the microprocessor 42 may also be communicably coupled to a storage device 46, which stores program instructions for execution by microprocessor 42 to perform the aforementioned pre-processing and, optionally, disease state determination. Microprocessor 42 may also be communicably coupled to an output interface 46 to allow downloading of the stored EEG data and to facilitate updating of the program instructions stored on storage device 46. The interface 48 may be a wired or wireless interface. For example, the interface 48 may be in the form of a jack adapted to receive a plug for wired connection to a receiving unit, or a transmitter adapted for radio frequency communication with a receiving unit, in which case any of several kinds of radio frequency communications may be used, for example, Bluetooth, Bluetooth Low Energy (BLE), Zigbee, infrared, WiFi HaLow (IEEE 802.22h), Z-wave, Thread, SigFox, Dash7, or other form of radio frequency communication. Power for the unit may be provided by a battery 50.

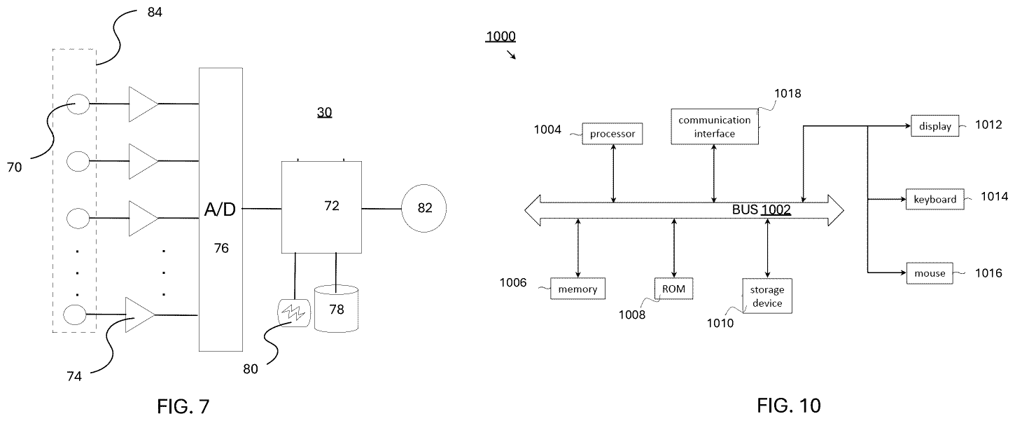

In various embodiments, the wearable device 30 may be in the form of a helmet to be worn by a patient, or may be configured in another arrangement. The sensors of the electronic controller section 32 may be moveable with respect to the patient so as to permit locating the sensors at different positions on the wearer. In various embodiments, 19, 32, or 64 sensors may be used, although in some cases different numbers of sensors may be employed. For example, as shown in FIG. 7, one arrangement of the wearable device 30 may be in the form of headband 84 with sensors 70 tethered by wires so as to be independently positionable on the patient's scalp. The sensors and associated electronics may allow for detection of electrical signals. Sensors 70 may be attached by electrical leads to a processor 72 (programmed in the fashion described above), e.g., via associated amplifiers 74 and analog-to-digital converter(s) 76, etc., which samples the signals from the sensors periodically. A record of the sampled signals may be stored locally, e.g., in a suitable writable memory 78 such as a flash memory, and also may be transmitted to a remote monitoring location via a telemetry transmitter 80 and an associated antenna. Alternatively, the telemetry may be transmitted only when the transmitter is activated, e.g., by an on-scene health care professional, or by the wearer him/herself. Upon command, any stored samples may be similarly transmitted so that a history of the wearer's biomarkers and, optionally, vital signs can be analyzed by a physician or other person at the remote monitoring station, or locally via an output port 82. The sensors 70 may be part of a head worn unit 84 or may be individually provided.

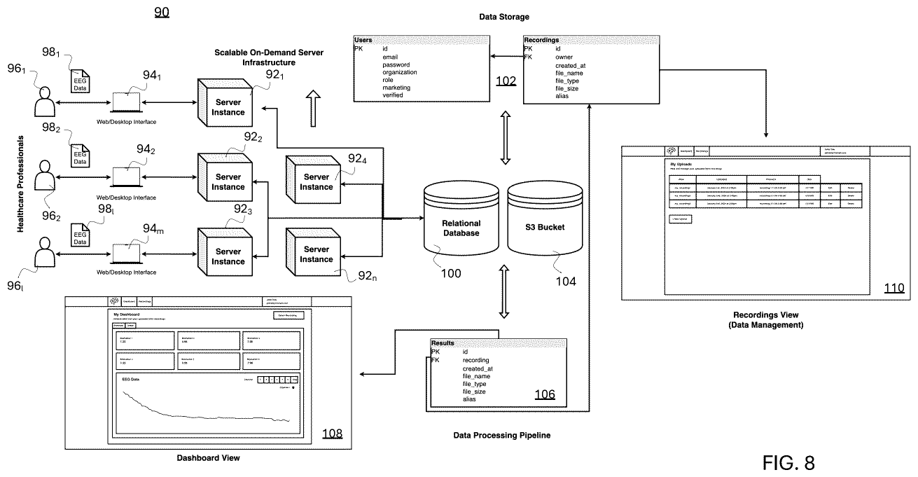

Referring now to FIG. 8, a system 90 for providing software-as-a-service access to server instances 92₁-92ₙ configured for assessing neurological conditions from EEG data is illustrated. Each server instance 92₁-92ₙ may be instantiated on-demand as client demands for services are received. For example, each server instance may be instantiated as an application running on appropriate computer hardware, as described below, or as an application running on a virtual machine that is running on such hardware. A load balancing appliance (not shown) may assess the need to distribute client workload across server instances and call for additional server instances to be instantiated as necessary to meet workload and performance requirements. During periods of reduced client demand, server instances may be decommissioned so as not to incur unnecessary expense for the service provider and/or to free up computer hardware resources for other purposes.

As illustrated, each server instance is configured to service one or more clients 94₁-94ₘ. The clients may be computer systems or mobile device, such as a smart phone or tablet, systems deployed at sites remote from the server instances and communication between the clients and the server instances may be facilitated through one or more network connections, such as connections over TCP/IP networks (e.g., the Internet), or similar means. In some cases, clients may be co-located with server instances, for example where a server instance is instantiated on the same device as a client, or where the server instance is instantiated on a local area network that also includes one or more clients. The clients are used by health care providers 96₁-96ₗ to upload EEG data 98₁-98ₗ that is captured using sensor arrangements in the fashion described above. The clients may provide the EEG data through the facilities of a user interface that runs in a web browser (e.g., as a web page provided to the client by a server instance), or as a desktop or other application on the client.

Each server instance 92₁-92ₙ may be configured to provide the data preprocessing for EEG data uploaded by a client 94₁-94ₘ in the manner described above. That is, the server instances may be configured to provide the band pass filtering, bad channel removal, artifact removal, ocular artifact detection, and feature extraction discussed above in connection with FIG. 1. Alternatively, in cases where clients run appropriate application, the client may perform the preprocessing and provide pre-processed EEG data to the server instance. Additionally, the server instances 92₁-92ₙ may be configured as machine learning engines with instances of trained models to determine the presence or absence of disease states of interest based on features extracted from the uploaded EEG data. That is, the server instances may be provided with trained models that function as discriminators to provide outputs that represent the likelihood of disease state presence from collected EEG data. One such discriminator is a support vector machine. Other discriminators that may be used include decision tree models, random forest models, gradient descent learning models, logistic regression models, and recurrent neural network models. In some cases, server instances may be configured to subject the features extracted from the EEG data to multiple (e.g., two or more) discriminators and to compare and/or combine the prediction results of the multiple discriminators to arrive at a final prediction of disease state or not.

The EEG data, whether in raw form, preprocessed form, or both, may be stored 102 in a relational database 100 along with patient identifying information and the results of any data analysis, health care provider notes, etc. For example, a Simple Storage Service (S3) 104 provided by Amazon Web Services Inc. or a similar provider may be used as an object storage system to provide both scalability of the server instances and data storage and availability. Such a system affords the health care providers the ability to view stored data as well as processed EEG data and analysis results at a client, for example using a dashboard-like view 108. The dashboard view may be provided as part of a data processing pipeline 106, which includes the data upload, data pre-process, and data analysis flows. Additionally, data management views 108 may be provided to allow a health care provider access to historical EEG data captures and analysis results, e.g., as a means of tracking patient history and disease progression.

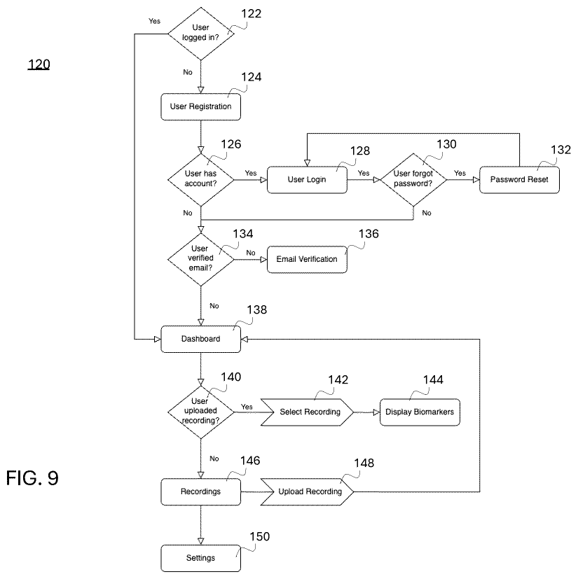

An example of a user session 120 for system 90 is shown in FIG. 9. Not all of the steps in session 120 may be performed for a given use of system 90, but they are being illustrated for purposes of a complete explanation. At 122, a check is made to determine if a user has logging in to system 90 via a client or other means. If an access is being attempted for the first time, the user may need to create an account and become a registered user before being allowed to use system 90. Alternatively, if the user already has an account but is unable to access it, an account recovery procedure may need to be invoked. In either case, a registration process 124 may be initiated and a query made as to the purpose of the user's visit. If the user has an existing account 126, but is unable to presently access it, the user is afforded an opportunity to recover a forgotten password or create a new one 128, 130, 132. For example, a log-in screen may be presented 128 and, if the user indicates he/she cannot remember the account password 130, a password reset option 132 is provided. On the other hand, if the problem is not a forgotten password, other means of verifying the user 134, for example, via email 136 or a second factor authentication may be provided.

Once a user has created an account, or a registered user has successfully logged-in and/or been verified, the user's dashboard view 108 may be presented 138 at the client. The dashboard view may provide the user the ability to perform account maintenance or other activities, upload new EEG data for pre-processing and analysis, or revisit previously uploaded EEG data for review, etc. For example, if the user selects an option to upload new EEG data 140, the data can be uploaded 142 as a file from the client or an associated wearable device such as those described above, preprocessing performed and the resulting biomarkers displayed for review 144. The displayed biomarkers may be provided in conjunction with an analysis result to indicate a disease state or not. If the user is not uploading new EEG data 146, the user may access previously recorded EEG data 146, e.g., from a data management view 110. Individual recordings may be reviewed 148 by the provider, for example in preparation for a patient consultation or other reason. Additionally, as part of a session, a user may revise or update various account settings 150.

As is apparent from the foregoing discussion, aspects of the present invention involve the use of various computer systems and computer readable storage media having computer-readable instructions stored thereon. FIG. 10 provides an example of a system 1000 that may be representative of any of the computing systems discussed herein, for example the servers and server instances described above. Note, not all of the various computer systems have all of the features of system 1000. For example, certain ones of the computer systems discussed above may not include a display inasmuch as the display function may be provided by a client computer communicatively coupled to the computer system or a display function may be unnecessary. Such details are not critical to the present invention.

System 1000 includes a bus 1002 or other communication mechanism for communicating information, and a processor 1004 coupled with the bus 1002 for processing information. Computer system 1000 also includes a main memory 1006, such as a random access memory (RAM) or other dynamic storage device, coupled to the bus 1002 for storing information and instructions to be executed by processor 1004. Main memory 1006 also may be used for storing temporary variables or other intermediate information during execution of instructions to be executed by processor 1004. Computer system 1000 further includes a read only memory (ROM) 1008 or other static storage device coupled to the bus 1002 for storing static information and instructions for the processor 1004. A storage device 1010, for example a hard disk, flash memory-based storage medium, or other storage medium from which processor 1004 can read, is provided and coupled to the bus 1002 for storing information and instructions (e.g., operating systems, applications programs and the like).

Computer system 1000 may be coupled via the bus 1002 to a display 1012, such as a flat panel display, for displaying information to a computer user. An input device 1014, such as a keyboard including alphanumeric and other keys, may be coupled to the bus 1002 for communicating information and command selections to the processor 1004. Another type of user input device is cursor control device 1016, such as a mouse, a trackpad, or similar input device for communicating direction information and command selections to processor 1004 and for controlling cursor movement on the display 1012. Other user interface devices, such as microphones, speakers, etc. are not shown in detail but may be involved with the receipt of user input and/or presentation of output.

The processes referred to herein may be implemented by processor 1004 executing appropriate sequences of computer-readable instructions contained in main memory 1006. Such instructions may be read into main memory 1006 from another computer-readable medium, such as storage device 1010, and execution of the sequences of instructions contained in the main memory 1006 causes the processor 1004 to perform the associated actions. In alternative embodiments, hard-wired circuitry or firmware-controlled processing units may be used in place of or in combination with processor 1004 and its associated computer software instructions to implement the invention. The computer-readable instructions may be rendered in any computer language.

In general, all of the above process descriptions are meant to encompass any series of logical steps performed in a sequence to accomplish a given purpose, which is the hallmark of any computer-executable application. Unless specifically stated otherwise, it should be appreciated that throughout the description of the present invention, use of terms such as “processing”, “computing”, “calculating”, “determining”, “displaying”, “receiving”, “transmitting” or the like, refer to the action and processes of an appropriately programmed computer system, such as computer system 1000 or similar electronic computing device, that manipulates and transforms data represented as physical (electronic) quantities within its registers and memories into other data similarly represented as physical quantities within its memories or registers or other such information storage, transmission or display devices.

Computer system 1000 also includes a communication interface 1018 coupled to the bus 1002. Communication interface 1018 may provide a two-way data communication channel with a computer network, which provides connectivity to and among the various computer systems discussed above. For example, communication interface 1018 may be a local area network (LAN) card to provide a data communication connection to a compatible LAN, which itself is communicatively coupled to the Internet through one or more Internet service provider networks. The precise details of such communication paths are not critical to the present invention. What is important is that computer system 1000 can send and receive messages and data through the communication interface 1018 and in that way communicate with hosts accessible via the Internet. It is noted that the components of system 1000 may be located in a single device or located in a plurality of physically and/or geographically distributed devices.

Thus, methods and systems for assessing neurological conditions have been described. It should be understood that the above-description is intended to be illustrative, and not restrictive. Many other embodiments will be apparent to those of skill in the art upon reviewing the above description.

What is claimed is:

1. A method for processing raw electroencephalogram (EEG) resting state data, the method comprising:
obtaining the raw EEG resting state data from a patient using one or more sensors affixed to the patient or a device worn by the patient;
band pass filtering the raw EEG data using a bandpass filter with a passband of approximately 0.5-32 Hz to obtain filtered EEG data, the filtered EEG data being made up of signals in a plurality of channels;
subjecting the filtered EEG data to bad channel removal so as to remove those of the signals in channels that exhibit poor signal-to-noise ratios, the poor signal-to-noise ratios being exhibited in a time domain by channel amplitudes that deviate by more than a specified amount and/or being exhibited in a frequency domain by deviations in channel spectral data;
following the bad channel removal, performing large artifact removal to remove artifacts from the filtered EEG data due to signals from electrical sources other than the patient's brain activity;
following the large artifact removal, extracting biomarkers from the filtered EEG data, wherein for each channel of the filtered EEG data the biomarkers include spectral features, statistical features, time series features, and graph features;
providing the extracted biomarkers as inputs to a machine learning engine that has been trained to distinguish disease states from non-disease states, and outputting from the machine learning engine a probability from 0 to 1 of the presence of a disease state as indicated by the extracted biomarkers.

2. The method of claim 1, wherein the bad channel removal is performed using one of: a k-nearest neighbor approach, anomaly detection, or a Local Outlier Probability (LoOP) approach.

3. The method of claim 1, wherein the large artifact removal is performed by eliminating channel data that exceeds a predefined amplitude threshold.

4. The method of claim 1 wherein the large artifact removal is performed by one or more of: regression methods, wavelet transformation methods, Blind Source Separation (BSS)-based methods, or Empirical-mode Decomposition (EMD) methods.

5. The method of claim 1, wherein the feature extraction is performed using one or more of: Fast Fourier Transforms (FFTs), discrete wavelet transform (DWT), time frequency distributions, eigenvector methods, or autoregressive methods.

6. The method of claim 1, wherein the spectral features comprise alpha peak frequency, band power, and delta/theta power ratio.

7. The method of claim 6, wherein the statistical features comprise Hjorth activity, Hjorth mobility, and Hjorth complexity.

8. The method of claim 7, wherein the time series features comprise normalized Lemple-Ziv complexity and fractal dimension.

9. The method of claim 8, wherein the graph features comprise mean jump length across a quantile graph (Delta score).

10. The method of claim 1, wherein the spectral features comprise alpha peak frequency, band power, and delta/theta power ratio; the statistical features comprise Hjorth activity, Hjorth mobility, and Hjorth complexity; the time series features comprise normalized Lemple-Ziv complexity and fractal dimension; and the graph features comprise mean jump length across a quantile graph (Delta score).

11. The method of claim 10, wherein the bad channel removal is performed using one of: a k-nearest neighbor approach, anomaly detection, or a Local Outlier Probability (LoOP) approach.

12. The method of claim 11, wherein the large artifact removal is performed by one or more of: eliminating channel data that exceeds a predefined amplitude threshold, regression methods, wavelet transformation methods, Blind Source Separation (BSS)-based methods, or Empirical-mode Decomposition (EMD) methods.

13. The method of claim 12, wherein the feature extraction is performed using one or more of: Fast Fourier Transforms (FFTs), discrete wavelet transform (DWT), time frequency distributions, eigenvector methods, or autoregressive methods.

14. The method of claim 1, wherein the machine learning engine instantiated as a server instance of a software-as-a-service platform.

15. The method of claim 1, wherein the machine learning engine is trained to determine the presence of Alzheimer's disease from a disease-free condition based on the biomarkers.

16. The method of claim 15, wherein the machine learning engine instantiated as a server instance of a software-as-a-service platform.

17. The method of claim 15, wherein the spectral features comprise alpha peak frequency, band power, and delta/theta power ratio; the statistical features comprise Hjorth activity, Hjorth mobility, and Hjorth complexity; the time series features comprise normalized Lemple-Ziv complexity and fractal dimension; and the graph features comprise mean jump length across a quantile graph (Delta score).

18. The method of claim 17, wherein the bad channel removal is performed using one of: a k-nearest neighbor approach, anomaly detection, or a Local Outlier Probability (LoOP) approach; the large artifact removal is performed by one or more of: eliminating channel data that exceeds a predefined amplitude threshold, regression methods, wavelet transformation methods, Blind Source Separation (BSS)-based methods, or Empirical-mode Decomposition (EMD) methods; and the feature extraction is performed using one or more of: Fast Fourier Transforms (FFTs), discrete wavelet transform (DWT), time frequency distributions, eigenvector methods, or autoregressive methods.

19. A wearable device comprising a processor and a memory coupled to the processor, the memory storing instructions which, when executed by the processor, cause the processor to execute the steps for processing raw EEG resting state data as in claim 1.

20. A computer system, comprising a processor and a memory coupled to the processor, the memory storing instructions which, when executed by the processor, cause the processor to execute the steps for processing raw EEG resting state data as in claim 1, and the server further comprising a communication interface configured to provide a two-way data communication channel with a computer network.
